(19) United States
(12) Patent Application Publication
(10) Pub. No.: US 2015/0184202 A1
(43) Pub. Date: Jul. 2, 2015

(54) Method for the Production of Diiobutene

(71) Applicant: Oxea GmbH, Oberhausen (DE)

(72) Inventors: Jens Klabunde, Dusseldorf (DE); Leif Johnen, Voerde (DE); Guido D. Frey, Riedstadt (DE); Sebastian Geisel, Essen (DE); Horst Lange, Bochum (DE); Heinz Strutz, Moers (DE)

(73) Assignee: Oxea GmbH, Oberhausen (DE)

(21) Appl. No.: 14/406,946

(22) PCT Filed: Jul. 1, 2013
(86) PCT No.: PCT/EP2013/063796

§ 371 Date: Dec. 10, 2014

(30) Foreign Application Priority Data
Jul. 2, 2012 (DE) 10 2012 105 877.6

(51) Int. Cl. C12P 5/02; C07C 2/08

(52) U.S. Cl. CPC C12P 5/026; C07C 2/08

(57) ABSTRACT

The present invention relates to a method of producing diisobutene from fermentative produced isobutene, wherein the higher purity of the isobutene improves the method and the properties of the produced diisobutene.

### CLAIM FOR PRIORITY

This application is a national phase application of PCT/EP2013/063796 FILED Jul. 1, 2013 which was based on application DE 10 2012 105 877.6 FILED Jul. 2, 2012. The priorities of PCT/EP2013/063796 and DE 10 2012 105 877.6 are hereby claimed and their disclosures incorporated herein by reference.

### TECHNICAL FIELD

The present invention relates to a method of producing diisobutene preferably from renewable raw materials.

### BACKGROUND

Diisobutene (2,4,4-trimethyl-1-pentene and 2,4,4-trimethyl-2-pentene as the main components) is an important industrial chemical and an important intermediate product in the production of other major industrial compounds. For example, diisobutene will be processed into isononanal, isononanol, isononanoic acid and derivatives of these oxo chemicals extended by one carbon atom (Ullmanns Encyklopadie der technischen Chemie, 4th edition, 1975, Verlag Chemie, Volume 9, pages 143-145).

Processes for preparing diisobutene are known for some time and are described inter alia in Baerns et. al. Technische Chemie, 1st edition, Wiley-VCH, Weinheim 2006. Usually one starts from isobutene obtained from raffinate I, which acid-catalysed is dimerised into diisobutene. Because of the immense importance of diisobutene for the industrial chemistry, however, it is constantly searched for further improvements with respect to alternative methods and alternative sources of raw materials for the production of diisobutene.

The use of renewable raw materials as starting materials for the production of organic chemicals on an industrial scale is becoming increasingly important. On the one hand ressources based on petroleum, natural gas and coal should be conserved, and the other hand with renewable raw materials carbon dioxide is bound in an industrially usable carbon source, which is principally inexpensive and available in large quantities. Examples for the use of renewable raw materials for the industrial production of organic chemicals include the production of citric acid, 1,3-propanediol, L-lysine, succinic acid, lactic acid, and itaconic acid.

Renewable raw materials are not yet used for the production of diisobutene. Thus, the task will be to provide an alternative improved method for the production of diisobutene preferably from sources of renewable raw materials. Herein it is of particular importance with regard to the use of diisobutene that preferably isomer-free isobutene is used for the production of diisobutene.

### SUMMARY OF INVENTION

The object of providing an alternative, improved method for the production of diisobutene preferably from sources of renewable raw materials is achieved by a method of producing diisobutene comprising the steps of:
a) fermentative preparation of isobutene;
b) dimerisation of isobutene into diisobutene;
c) purifying the diisobutene.

It surprisingly has been found that fermentatively produced isobutene is of such a high purity with respect to linear butene isomers that the subsequent acid-catalysed dimerisation delivers diisobutene in a high purity and yield. In the prior art method are known, in which isobutene in high purity is obtained biochemically on a laboratory scale. Thus, however, starting from the direct precursor 3-hydroxyisovaleriat (3-hydroxy-3-methylbutyrate), Gogerty, D. S. and Bobik, T. A. 2010, Applied and Environmental Microbiology, pages 8004-8010, investigated the fermentative-enzymatic synthesis of isobutene, wherein according to GC no significant amounts of n-butene isomers were revealed in the valuable product.

The by-product carbon dioxide formed during the fermentation and optionally other inert gases may optionally be removed by suitable separation techniques in a conventional manner. In most embodiments of the invention the conversion of isobutene into diisobutene can be carried out even without further prior purification of the isobutene, thus illustrating a preferred embodiment of the invention. In this embodiment of the invention the fermentative process of the invention takes advantage of the high selectivity to isobutene as C₄-olefin. On the other hand carbon dioxide and other inert gases do not disturbe the dimerisation of isobutene into diisobutene. In particular cases, however, it may be appropriate to initially separate carbon dioxide and other inert gases from the isobutene.

### DETAILED DESCRIPTION

The term “fermentative production” of isobutene means particularly that isobutene is derived either
by means of microorganisms, preferably from renewable raw materials; and/or
by a cell-free enzymatic process, also preferably from renewable raw materials.

Isobutene is—as far as is known—not a natural product in the sense that it is formed in metabolic processes in organisms in such amounts that an industrial use seems appropriate. However, isobutene is produced in very small amounts from naturally occurring microorganisms (U.S. Pat. No. 4,698,304; Fukuda, H. et al., 1984, From Agricultural and Biological Chemistry (1984), 48 (6), pp. 1679-82). Thus, in the previously known embodiments of the invention, the fermentative preparation of isobutene occurs by means of modified, non-natural microorganisms and the corresponding modified enzymes, respectively. Such microorganisms are disclosed in US 2011165644 (A1), wherein in Example 13 the synthesis of isobutene from glucose in suitable microorganisms is discussed. In WO 2012052427 and WO 2011032934 further enzymatic reactions are described, which describe the formation of isobutene as a series of sequential enzymatic syntheses

I) acetone into 3-hydroxyisovaleriate; and

II) 3-hydroxyisovaleriate into isobutene and carbon dioxide.

The enzymatically catalysed decomposition of 3-hydroxyisovaleriate into isobutene and carbon dioxide is also discussed in Gogerty, D. S. and Bobik, T. A., 2010, Applied and Environmental Microbiology, pages 8004-8010. Here, according to GC, no significant amounts of n-butene isomers were revealed in the valuable product. Even in aqueous, non-enzymatically catalysed systems one observes a spontaneous separation of carbon dioxide from 3-hydroxyisovaleriate under formation of isobutene, which further reacts with the present water in a balance reaction into tert-butanol (Pressman, D. and Lucas, H. J., 1940, Journal of the American Chemical Society, pages 2069-2081).

If this sequence of enzymatic syntheses described in I and II is included in a suitable microbial host organism which is capable of synthesizing acetone from metabolic precursors or to transport externally supplied acetone by means of a passive or active transport through the cell wall into the cell, by means of a non-natural microorganism derived in such a manner isobutene can be produced by a fermentative process with a good yield. Microorganisms that synthesize acetone from different carbohydrates have long been known and are described inter alia in Jones, T. D. and Woods, D. R., 1986, Microb. Reviews, pages 484-524. Taylor, D. G. et al., 1980, Journal of General Microbiology, 118, pages 159-170 describe microorganisms that use acetone as a sole carbon source and, thus, are able to transport acetone across the cell wall into the cell.

Another possible metabolic pathway proceeds via the reaction sequence:

I) pyruvate into 2-acetolactate;

II) 2-acetolactate into 2,3-dihydroxyisovaleriate;

III) 2,3-dihydroxyisovaleriate into 2-oxoisovaleriate;

IV) 2-oxoisovaleriate into isobutyraldehyde;

V) isobutyraldehyde into iso-butanol; and

VI) isobutanol into isobutene

and is described inter alia in WO 2011076689 and WO 2011076691.

The term “diisobutene”—as already described—means 2,4,4-trimethyl-1-pentene, 2,4,4-trimethyl-2-pentene as the main components and any mixtures of these two compounds.

According to a preferred embodiment of the invention no purification of the isobutene is carried out between steps a) and b), in particular no purification to remove linear butene isomers and optionally inert gases such as carbon dioxide and/or nitrogen. “Purification” means in particular (but not limited thereto) the following methods:
Distillation processes (which, however, are complicated by the fact that the separation of linear butene isomers occurring in the overall process requires a lot of effort, since the boiling points of the isomers are very close to each other, see Kirk-Othmer, Encyclopedia of Chemical Technology, 3rd edition, 1978, vol. 4, John Wiley & Sons Inc., pp. 358-360).
Purification or separation methods in which isobutene is separated due to the increased chemical reactivity by means of a chemical reaction, and then is converted back into isobutene. This includes methods such as reversible proton-catalysed water addition to tert-butanol or the methanol addition to methyl-tert-butylether (see EP 1489062). From these adducts then isobutene is recovered by a reverse reaction (see Weissermel, Arpe, Industrielle Organische Chemie, VCH Verlagsgesellschaft, 3rd edition, 1988, pp. 74-79).
Purification or separation methods in which isobutene is separated from linear butene isomers due to the more compact spatial molecular structure by means of suitable physical size exclusion methods, for example, by means of molecular sieves having an appropriate pore size (see W O2012040859, Weissermel, Arpe, Industrielle Organische Chemie, VCH Verlagsgesellschaft, 3rd edition, 1988, p.74).
Purification and separation methods suitable for the removal of carbon dioxide.

According to a preferred embodiment of the invention, the isobutene is derived in step a) from trisaccharides, disaccharides, monosaccharides, acetone or mixtures thereof. The tri- and di-saccharides used are in particular raffinose, cellobiose, lactose, isomaltose, maltose and sucrose. The monosaccharides used are in particular D-glucose, D-fructose, D-galactose, D-mannose, DL-arabinose and DL-xylose. Herein the tri-, di-and monosaccharides inter alia originate (but not limited thereto)
from the digestion and the depolymerization of cellulose and hemicellulose using appropriate methods;
directly from plants with high sugar content such as sugar beet, sugar cane, palm sugar, maple sugar, sorghum, silver date palm, honey palm, Palmyra palm and agaves by means of extraction;
from the depolymerization of plant starch by hydrolysis;
from the depolymerization of animal glycogen by hydrolysis;
directly from milk obtained from the dairy industry.

In a further preferred embodiment of the invention exclusively renewable raw materials are used for the fermentative production of isobutene. If desired, the origin of the carbon atoms derived from sources of renewable raw materials can be determined by the test method described in ASTM D6866. Herein the ratio of C¹⁴ to C¹² carbon isotopes is determined and compared with the isotopic ratio of a reference substance, the carbon atoms of which originate at 100% from sources of renewable raw materials. This test method is also known in a modified form as radiocarbon method and is described among others in Olsson, I. U., 1991, Euro Courses: Advanced Scientific Techniques, volume 1, Issue Sci. Dating Methods, pages 15-35.

According to a preferred embodiment of the invention the fermentation process is carried out at temperatures of ≧20° C. to ≦45° C. and under atmospheric pressure, wherein isobutene is released as a gaseous product. This embodiment has the advantage that the thus obtained isobutene can be reused directly or after separation of inert gases.

Alternatively, the fermentation process is performed at temperatures of ≧20° C. to ≦45° C. and under a pressure between 1 to 30 bar in accordance with a likewise preferred embodiment of the invention. In this case isobutene can be obtained as a liquid compound and be separated directly from the fermentation medium by phase separation. In this preferred embodiment the separation of inert gases can be considerably facilitated.

According to a preferred embodiment step b) is carried out under acid catalysis. Herein, for example, sulfuric acid or acidic ion exchangers come into consideration, as described in Weissermel, Arpe, Industrielle Organische Chemie, VCH Verlagsgesellschaft, 3rd edition, 1988, p. 77; Hydrocarbon Processing, April 1973, pp. 171-173. Alternatively the methods described in US 2004/0054246, U.S. Pat. No. 4,100,220 (A), U.S. Pat. No. 4,447,668 (A) and U.S. Pat. No. 5,877,372 (A) can be used.

The method comprises a further step c) which is performed subsequently to b):
c) Purification of the diisobutene, preferably by distillation.

Step c) is preferably carried out such that the unreacted volatile components are separated from the diisobutene and the diisobutene obtained is purified by distillation from the triisobutene which may be formed in small amounts and higher isobutene oligomers. The thus obtained triisobutene, and the thus obtained higher isobutene oligomers may also be refined into valuable secondary products.

The diisobutene produced in this manner can be processed further in subsequent reactions into isononyl derivatives, for example in accordance with the hydroformylation reaction or the Koch reaction (Ullmanns Encyclopadie der technischem Chemie, 4th edition, 1975, Verlag Chemie, Volume 9, pages 144-145).

The synthesis steps to be used in accordance with the present invention, which are mentioned above and claimed and described in connection with the exemplary embodiments, are not subjected to particular exceptions with respect to their technical concept, such that the selection criteria known in the field of application can be applied without restriction.

The individual combinations of constituents and features of the embodiments mentioned above are exemplary, the exchange and substitution of these teachings with other teachings that are contained in this document with the references cited are also explicitly contemplated. Those skilled in the art will recognize that variations, modifications and other embodiments, which are described herein, may also occur without departing from the spirit and scope of the invention. Accordingly, the above description is to be considered exemplary rather than limiting. Terms such as “comprise” or “include” used in the claims do not exclude other constituents or steps. The indefinite article “a” does not exclude the meaning of a plural. The mere fact that certain amounts are recited in mutually different claims does not imply that a combination of these amounts cannot be employed to advantage. The scope of the invention is defined in the following claims and the associated equivalents.

1. Method of producing diisobutene, comprising the steps of
a) fermentative preparation of isobutene;
b) dimerisation of isobutene into diisobutene;
c) purifying the diisobutene.

2. Method according to claim 1, wherein between steps a) and b) no purification of the isobutene is carried out.

3. Method according to claim 1, wherein the isobutene in step a) is obtained from trisaccharides, disaccharides, monosaccharides, acetone or mixtures thereof

4. Method according to claim 1, wherein renewable raw materials are used for the fermentative production of isobutene.

5. Method according to claim 1, wherein the fermentation process is carried out at temperatures of ≧20° C. to ≦45° C. and under atmospheric pressure, and isobutene is released as a gaseous product.

6. Method according to claim 1, wherein the fermentation process is carried out at temperatures of ≧20° C. to ≦45° C. and under a pressure between 1 to 30 bar.

7. Method according to claim 1, wherein step b) is carried out under acid catalysis.

8. Method according to claim 1, wherein step c) is carried out by distillation.

9. Method according to claim 1, further comprising using the diisobutene for the preparation of derivatives which include compounds with the 3,5,5-trimethylhexyl or 2,2,4,4-tetramethylpentyl residue as a main component.

10. Method according to claim 2, wherein the isobutene in step a) is obtained from trisaccharides, disaccharides, monosaccharides, acetone or mixtures thereof.

11. Method according to claim 2, wherein renewable raw materials are used for the fermentative production of isobutene.

12. Method according to claim 2, wherein the fermentation process is carried out at temperatures of ≧20° C. to ≦45° C. and under atmospheric pressure, and isobutene is released as a gaseous product.

13. Method according to claim 2, wherein the fermentation process is carried out at temperatures of ≧20° C. to ≦45° C. and under a pressure between 1 to 30 bar.

14. Method according to claim 2, wherein step b) is carried out under acid catalysis.

15. Method according to claim 2, wherein step c) is carried out by distillation.

16. Method according to claim 1, further comprising using the diisobutene for the preparation of derivatives which include compounds with the 3,5,5-trimethylhexyl or 2,2,4,4-tetramethylpentyl residue as a main component.

17. Method according to claim 3, wherein the fermentation process is carried out at temperatures of ≧20° C. to ≦45° C. and under atmospheric pressure, and isobutene is released as a gaseous product.

18. Method according to claim 3, wherein the fermentation process is carried out at temperatures of ≧20° C. to ≦45° C. and under a pressure between 1 to 30 bar.

19. Method according to claim 3, wherein step b) is carried out under acid catalysis.

20. Method according to claim 3, wherein step c) is carried out by distillation.
